(19) United States
(12) Patent Application Publication
(10) Pub. No.: US 2007/0202574 A1
(43) Pub. Date: Aug. 30, 2007

(54) Novel Saccharothrix Strain an Antibiotics Derived Therefrom, i.e. Mutactimycins and Aldgamycins

(75) Inventors: Abdelghani Zitouni, Alger (DZ); Nasserdine Sabaou, Alger (DZ); Florence Mathieu, Escalquens (FR); Ahmed Lebrihi, Noueilles (FR)

Correspondence Address: FOLEY AND LARDNER LLP;SUITE 500, 3000 K STREET NW, WASHINGTON, DC, 20007, US

(73) Assignee: Institut National Polytechnique de Toulouse

(21) Appl. No.: 11/597,638

(22) PCT Filed: May 25, 2005
(86) PCT No.: PCT/FR05/01291

§ 371 Date: Feb. 6, 2007

(30) Foreign Application Priority Data
May 26, 2004 (FR) 0405659

(51) Int. Cl. C12P 19/56; C12N 1/20; C07H 15/24; A61K 31/7048; A61K 31/704

(52) U.S. Cl. 435/77; 435/78; 435/252.3; 536/6.4; 536/7.1; 514/28; 514/34

(57) ABSTRACT

The invention relates to a novel strain Saccharothrix actinomycete SA 103 deposited at CNCM on 16 Feb. 2004, number 1-3160 or a mutant strain thereof; a method and a medium for selection of said strain; and a method for the production of a broth, active concentrate and active compounds from a culture of said strain SA 103. The invention also relates to active compounds which can be obtained by the production method, i.e. novel mutatimycins and aldgamycins, pharmaceutical compositions comprising said active compounds and the use thereof in medicine and phytopharmaceuticals.

The invention relates to a new actinomycete strain Saccharothrix SA 103 registered with the CNCM on 6 Feb. 2004 under number I-3160 or a mutant strain thereof, a method and a medium for selecting the said strain, a method for producing a culture medium, an active concentrate and active compounds from a culture of the said strain SA 103. The invention also relates to the active compounds capable of being obtained by the production method, particularly novel mutatimycins and aldgamycins, pharmaceutical compositions comprising these active compounds and their use in the medical and phytopharmaceutical field.

Antimicrobial agents are widely used in many fields: human health, veterinary medicine, phytopathology, the food industry, leather and wood treatment, etc. Most of these agents are produced by microorganisms, whereof actinomycetes are among the most important.

Novel antimicrobial agents are always being sought, particularly antibacterial antibiotics.

In fact, it is known that by the very nature of the bacteria, they proliferate exponentially, and thereby increase their chances of becoming resistant to antibiotics, due to genetic mutations.

The inadequate use of antibiotics in the animal field, particularly human, or plant, and also in the food industry or the leather industry, increases the mutational capacity of the bacteria, and hence their resistance to antibiotics.

One solution to this problem is obviously to administer the antibiotics judiciously, but this solution may only be preventive.

A need therefore exists in the present state of the art to develop novel antibiotics. For this purpose, researchers have focused on extremophilic microorganisms.

The inventors have thereby isolated a novel actinomycete strain Saccharothrix SA 103 present in the sub-Saharan soil of Algeria, and have registered it with the Collection Nationale de Cultures de Microorganisms (CNCM) on 16 Feb. 2004 under number I-3160.

They have also discovered that this strain produced active compounds of the class of anthracyclines, some of which have never been previously identified, particularly mutactimycins (PR, F, G), and also active compounds of the class of anthracylines, particularly aldgamycins (G, H and P10b). These active compounds have antibacterial activity, particularly against gram positive bacteria, and also antiviral, antiproliferative and anticancer activity.

They therefore developed a method for obtaining these compounds from the strain Saccharothrix SA 103 registered with the CNCM on 16 Feb. 2004 under number I-3160, the said method having the advantage of being simple to implement. Thus these antibiotic compounds obtained from the strain Saccharothrix have a real pharmaceutical interest, both for humans and for animals, and a plant health interest, and can be produced on the industrial scale.

This is precisely the object of the present invention.

Thus, the primary subject of the invention is the actinomycete strain Saccharothrix SA 103 registered with the CNCM on 16 Feb. 2004 under number I-3160 or a mutant strain thereof.

In the context of the present application, “mutant strain”, “mutant”, “variant strain” or “variant”, equally mean a strain of Saccharothrix obtainable by selective mutation from the strain Saccharothrix SA 103 by preserving the capacity to produce at least one of the active compounds described below. The mutation techniques are known to a person skilled in the art and consist in placing the strain Saccharothrix SA 103 in the presence of a physical mutagenic agent, such as radiation, or a chemical mutagenic agent, for example acriflavin, then selecting in an appropriate medium the remaining mutants of interest by using their antibiotic spectrum (microorganism inhibition method as, for example, the minimum inhibiting concentration method or MIC, etc.).

The characteristics of the strain Saccharothrix SA 103 according to the invention are described in the part “Examples” of the present application.

The strain Saccharothrix SA 103 according to the invention or one of its mutants is cultured in a medium containing a variety of nutrient substances generally used for the growth of actinomycetes. For example, as a carbon source, use can be made of glucose, glycerin, sucrose, starch, maltose, or animal or vegetable oils. As a nitrogen source, use can be made for example of organic nitrogen such as soybean flour, meat extracts, yeast extract, a peptone, maize maceration water, cotton cake or fish flour. Use can be made of inorganic nitrogen such as, for example, ammonium sulphate, ammonium chloride, sodium nitrate or ammonium phosphate. If necessary, additions can be used of sodium chloride, potassium chloride, potassium phosphate or divalent metallic salts such as Mg⁺⁺, Ca⁺⁺, Zn⁺⁺, Fe⁺⁺, Cu⁺⁺, Mn⁺⁺, or Ni⁺⁺, and amino acids or vitamins. Inclined geloses can be used.

A further subject of the present invention is a method for selecting the actinomycete strain Saccharothrix SA 103 according to the invention and/or at least one of its mutant strains, characterized in that it comprises the following steps:
a) contacting of a biological sample likely to contain the said strain and/or at least one of its mutant strains with an appropriate selection medium;
b) isolation of the said strain and/or at least one of its mutant strains.

A further subject of the present invention is an appropriate selection medium for isolating the actinomycete strain Saccharothrix SA 103 according to the invention and/or at least-one of its mutant strains.

In the present application, appropriate selection medium means any medium by means of which at least the actinomycete strain Saccharothrix SA 103 according to the invention is capable of developing but which prevents the growth of at least one strain different from the strain Saccharothrix SA 103 or of another microorganism such as a fungus; preferably, the selection medium according to the invention serves to prevent the growth of all strains different from the strain Saccharothrix SA 103 according to the invention or of a mutant strain thereof.

Such a selection medium comprises a variety of nutrient substances as defined above and at least one selective product such as, for example, an antibacterial agent to which only the strain Saccharothrix SA 103 according to the invention and/or at least one of its mutant strains is resistant, and/or an antifungal agent to eliminate the fungi whereof the rapid growth could hinder or even prevent the strain of the invention from growing.

As an example of an appropriate selection medium for isolating the strain Saccharothrix SA 103 according to the invention, mention can be made of the M1S medium which corresponds to a humic-vitamin B gelose also comprising a lysozyme, preferably at a proportion of about 0.005%, cycloserine, preferably at a rate of about 10 mg/l, the pH of this medium being in the range of about 8.5 to about 9.

Another preferred example of an appropriate selection medium for isolating the strain actinomycete SA 103 according to the invention is the HV-Vitamin B medium also comprising lysozyme, preferably at the rate of about 0.005%, cycloserine, preferably at the rate of about 10 mg/l, penicillin, preferably at the rate of about 10 mg/l, and rifampicin, preferably at the rate of about 5 mg/l, the pH medium being in the range of about 8.5 to about 9.

The selectivity of the appropriate selection medium of the invention can be reinforced by adding to said medium other selective products to which the strain SA 103 according to the invention is resistant, such as particularly sodium azide, preferably at the rate of about 0.001%, potassium tellurite, preferably at the rate of about 0.01%, and/or crystal violet, preferably at the rate of about 0.001%.

As an example of an antifungal agent which can be added to the appropriate selection medium of the invention, mention can be made of actidione and/or nystatin.

When selecting a mutant strain of the strain Saccharothrix SA 103 according the to the invention, the selective medium may also,.consist of a culture medium to which a single amino acid has been added, as well known to a person skilled in the art.

The strain SA 103 of the invention which the inventors have succeeded in isolating is an extremely rare strain. In fact, a single colony has been identified in more than 120 samples analysed and with or without selective products such as, for example, the antibiotics mentioned above.

A further subject of the present invention is a method for producing a culture medium from a culture of the actinomycete strain Saccharothrix SA 103 and/or at least one of its mutant strains according to the invention, characterized in that it comprises the following steps:
a) fermentation of the said strain in a nutrient medium to obtain the culture medium;
b) optionally, separation of the culture medium obtained in step a).

Aerobic fermentation in a liquid medium is preferred, as in the case of the production of other antibiotics, and the production of the active compounds from the strain Saccharothrix SA 103 according to the invention can be carried out at any temperature favourable to the growth of this strain, that is, ranging from ambient temperature to 43° C. Preferably, use is made of a temperature of between 25° C. and 32° C. Even more preferably, the temperature is 30° C. This culture can last several days, for example from 2 to 10 days. Normally, the pH is slightly alkaline but the exact pH may vary according to the culture medium used. The strain is taken after growth on gelose, particularly inclined, and optionally stored at low temperature, and inoculated into a conventional liquid medium consisting of nutrient substances similar to those described above for the growth of the strain Saccharothrix SA 103 according to the invention, with stirring in order to obtain a culture medium.

The fermentation can be carried out in Erlenmeyer flasks and in industrial or laboratory fermentors having various capacities. When fermentation is carried out in a vessel, it is advisable to produce an inoculum in a nutrient culture by inoculating the nutrient culture with a sampling of the culture inclined or flat, or a freeze dried culture of the organism. After having obtained an inoculum in this way, it is transferred aseptically to the medium of the fermentation vessel for large scale production of the active compounds. The medium in which the inoculum is produced may be the same or may be different from the one used in the vessel, insofar as appropriate growth of the microorganism is obtained.

The nutrient medium of the invention generally contains the same types of nutrient substances as those of the culture medium of the strain Saccharothrix SA 103 according to the invention as described above (carbon source, nitrogen source, etc.). It may also contain antifoaming agents such as liquid paraffin, soybean oil, greases or silicone.

The optional step b) for separating the culture medium can be carried out by any method well known to a person skilled in the art;, alone or combined with another separation method, such as, for example, centrifugation, filtration or pasteurisation.

Thus preferably, the method for producing a culture medium according to the invention is characterized in that the separation carried out in optional step b) is a centrifugation and/or a filtration and/or a pasteurization.

A further subject of the invention is a culture medium which can be obtained by the inventive method.

Using the culture medium obtained by the production method of the invention, the active compounds are extracted. For this purpose, an organic solvent is used, such as for example, but without limitation, n-butanol, ethyl acetate, dichloromethane, n-propanol or 2-propanol.

After the extraction step, the organic phase can be dehydrated optionally to remove numerous polar impurities in the active concentrate, thereby subsequently facilitating the final purification of the active concentrate by HPLC. For this purpose, use can be made of anhydrous sodium sulphate or magnesium sulphate, and/or drying in vacuo of the organic phase. The use of filtration on gel (cross-linked dextran gel), cellulose column chromatography, an ion exchange resin or thin layer chromatography (silica gel) can also be considered.

Such methods are well known to a person skilled in the art, who knows how to use the various techniques alone or in combination, in the most suitable way in order to recover an active concentrate from the culture medium of the strain Saccharothrix SA 103 of the invention.

Thus, a further subject of the present invention is a method for producing an active concentrate from the culture medium according to the invention, characterized in that it comprises the following steps:
a) organic extraction of the culture medium with an organic solvent;
b) optionally, dehydration of the organic phase obtained and/or drying in vacuo;
c) optionally, placing of the active concentrate in suspension, preferably filtration of the suspension obtained, and repetition of the steps a) and b) of organic extraction and dehydration.

The active concentrate which can be obtained by the method of the present invention is a further subject of the present invention.

The active concentrate thereby obtained is then identified by means of instrumental analyses such as visible/ultraviolet absorption spectrum, the infrared absorption spectrum, the ¹H-NMR spectrum and the ¹³C-NMR spectrum, mass spectrometry, and also chromatographic analysis (silica gel or dextran gel chromatographic columns, ion exchange resins, liquid phase chromatography, reverse phase high performance liquid chromatography or HPLC, etc.). This serves to characterise and produce the various active compounds, also called “active fractions” which may be present in the active concentrate obtained.

Thus a further subject of the invention is a method for producing an active compound from the active concentrate according to the invention by reverse phase high performance liquid chromatography (reverse phase HPLC), preferably preceded by thin layer chromatography and/or low pressure liquid chromatography.

The active compounds which can be obtained by the production method of the invention hence correspond to eluates obtained by HPLC chromatography and are virtually pure.

Purification by HPLC chromatography is indispensable for separating the active compounds from the active concentrate. Thin layer chromatography and/or low pressure liquid chromatography such as of the Sephadex LH 20 type serves to obtain-an active concentrate stripped of numerous impurities and thereby facilitates the subsequent purification by HPLC chromatography.

Preferably, the method for producing an active compound according to the invention is characterized in that the active compound is a mutactimycin such as mutactimycin P11, mutactimycin PR, mutactimycin G or mutactimycin F, or an aldgamycin such as aldgamycin G, aldgamycin H or aldgamycin P10b, or the pharmaceutically acceptable addition salts, isomers, enantionmers, diastereoisomers, and mixtures of these active compounds.

Mutactimycin P11 has the following formula:

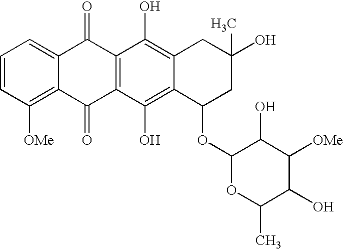

It has been identified as correspondent to mutactimycin C, already known and registered in the “Registry” base under number RN 138689-81-3.

Mutactimycin PR has the following formula:

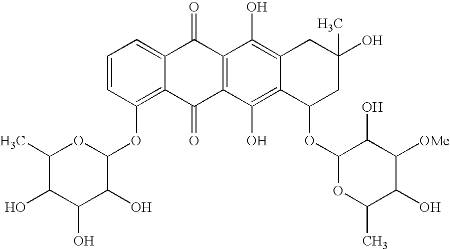

Mutactimycin F has the following formula:

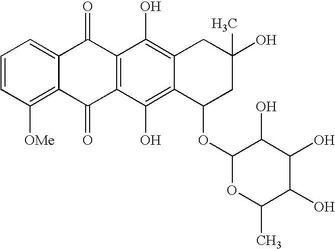

Mutactimycin G has the following formula:

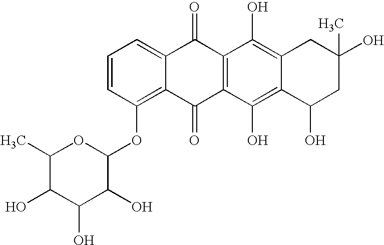

Aldgamycin G (or P10a) has the following formula:

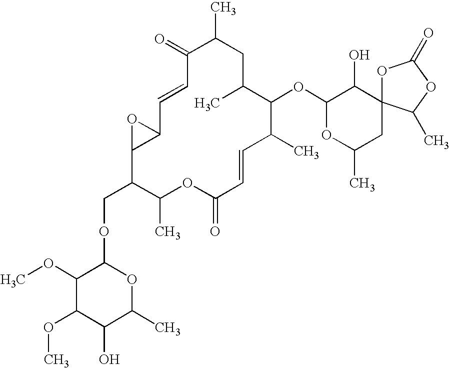

Aldgamycin with the following stereochemical formula is already known, registered in the “Registry” base under number RN 107745-56-2.

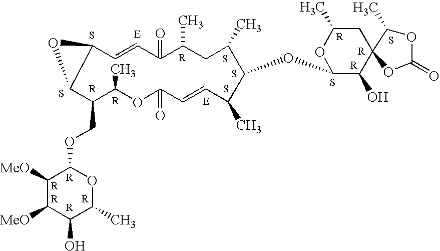

Aldgamycin H (or P8) has the following formula:

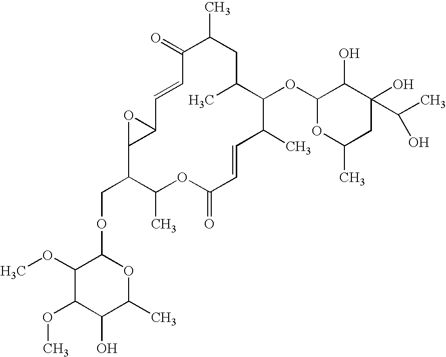

Aldgamycin P10b (or swalpamycine B) has the following formula:

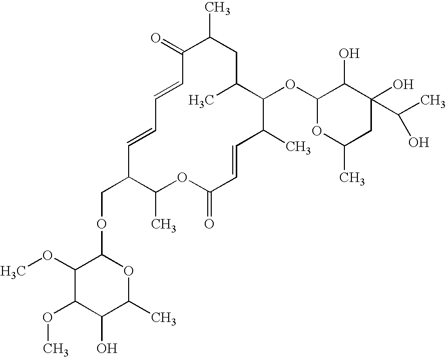

A further subject of the invention is an active compound capable of being obtained by the production method according to the invention, characterized in that the active compound is mutactimycin PR having the following formula:

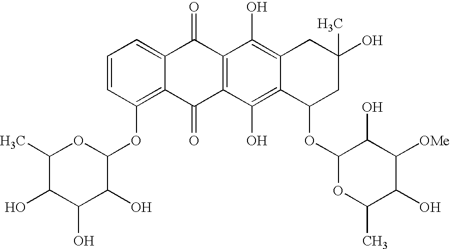

or its pharmaceutically acceptable addition salts, isomers, enantiomers, diastereoisomers, and mixtures thereof.

A further subject of the invention is an active compound capable of being obtained by the production method according to the invention, characterized in that the active compound is mutactimycin F having the following formula:

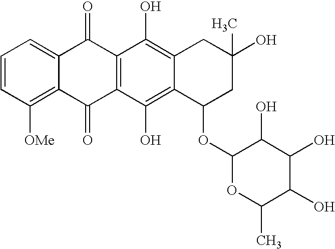

or its pharmaceutically acceptable addition salts, isomers, enantiomers, diastereoisomers, and mixtures thereof.

A further subject of the invention is an active compound capable of being obtained by the production method according to the invention, characterized in that the active compound is mutactimycin G having the following formula:

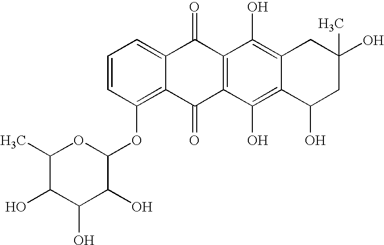

or its pharmaceutically acceptable addition salts, isomers, enantiomers, diastereoisomers, and mixtures thereof.

A further subject of the invention is an active compound capable of being obtained by the production method according to the invention, characterized in that the active compound is aldgamycin G having the following formula:

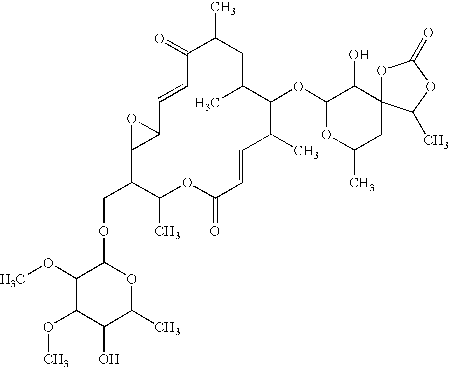

or its pharmaceutically acceptable addition salts, isomers, enantiomers, diastereoisomers, and mixtures thereof,

with the exception of aldgamycin G having the following stereochemical formula:

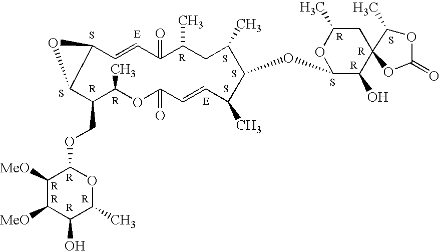

A further subject of the invention is an active compound which can be obtained by the production method of the invention, characterized in that the aldgamycin G has the following stereochemical formula:

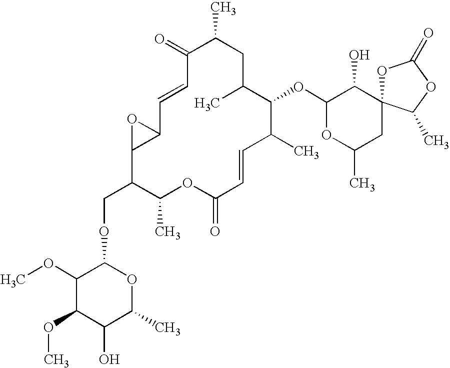

or its pharmaceutical acceptable addition salts, isomers, enantiomers, diastereoisomers, and mixtures thereof.

A further subject of the invention is an active compound capable of being obtained by the production method according to the invention, characterized in that the active compound is aldgamycin H having the following formula:

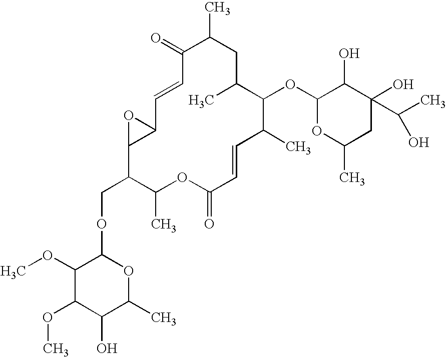

or its pharmaceutically acceptable addition salts, isomers, enantiomers, diastereoisomers, and mixtures thereof.

Preferably, the aldgamycin H has the following stereochemical formula:

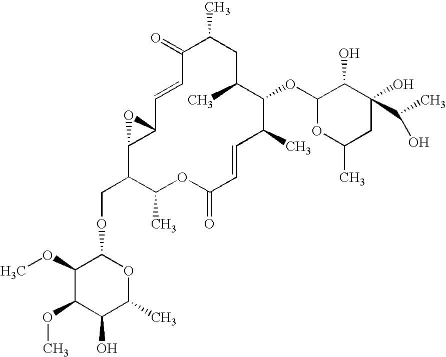

or its pharmaceutical acceptable addition salts, isomers, enantiomers, diastereoisomers, and mixtures thereof.

A further subject of the invention is an active compound capable of being obtained by the production method according to the invention, characterized in that the active compound is aldgamycin P10b having the following formula:

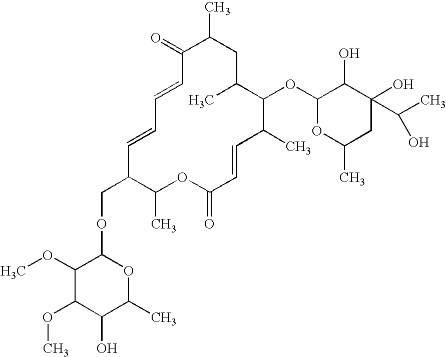

or its pharmaceutically acceptable addition salts, isomers, enantiomers, diastereoisomers, and mixtures thereof.

Preferably, the aldgamycin P10b has the following stereochemical formula:

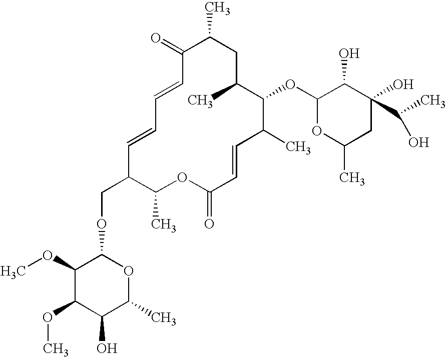

or its pharmaceutical acceptable addition salts, isomers, enantiomers, diastereoisomers, and mixtures thereof.

The mutactimycin PR, the mutactimycin F, the mutactimycin G, the aldgamycin G, the aldgamycin H or the aldgamycin P10b according to the invention can be obtained any method known to a person skilled in the art, such as for example, chemical or enzymatic methods. Preferably, use is made of the production method comprising a fermentation step from Saccharothrix SA 103 according to the invention as described above.

A further subject of the present invention is an active mixture of at least two active compounds selected from mutactimycin P11, mutactimycin PR, mutactimycin F, or mutactimycin G, or an aldgamycin such as aldgamycin G, aldgamycin H or aldgamycin P10b.

Optionally, the active mixture according to the invention may contain at least two active compounds selected among mutactimycin P11, mutactimycin PR, mutactimycin F or mutactimycin G, or an aldgamycin such as aldgamycin G, aldgamycin H or aldgamycin P10b, and another active compound having antibacterial, antiviral, antiproliferative or anticancer activity known to a person skilled in the art (antibiotics, etc.).

The active compounds of the invention can be used alone or in combination with other microbial agents, for example to prevent the growth or reduce the number of gram positive bacteria. Thus these compounds are useful from the medical standpoint, particularly for an antibacterial, antiviral or anticancer purpose.

A further subject of the invention is a pharmaceutical composition containing a therapeutically effective quantity of an active compound according to the invention, such as mutactimycin PR, mutactimycin F, mutactimycin G or aldgamycin G, aldgamycin H or aldgamycin P10b and a pharmaceutically acceptable excipient.

The pharmaceutical compositions can be prepared in any pharmaceutically appropriate form for the administration mode. Examples of such compositions comprise solid compositions for oral administration such as tablets, capsules, pills, powders and granules, liquid compositions for oral administrations such as solutions, suspensions, syrups and preparations for parenteral administration such as sterile solutions, suspensions or emulsions.

The active compounds of the present invention form addition salts, if applicable pharmaceutically acceptable with reagents for forming such salts, well known to a person skilled in the art.

The preferred quantities of the active compounds of the present invention used may vary according to the particular active compound used, the particular composition formulated, the application mode and the particular site, the host and the disease to be treated. In general the active compounds of the invention are injected by the intraperitoneal, intravenous, subcutaneous or local method, or administered orally. Various factors which modify the action of the medicinal product are taken into account by a person skilled in the art, for example the age, body weight, sex, diet, administration time, excretion rate, condition of the patient, combinations of medicinal products, sensitivities to reactions and severity of the disease. Administration can be carried out continuously or periodically in the maximum dose tolerated.

It should be noted that for use as antibacterial agents, the active compounds are generally administered so that the concentration of the active ingredient is higher than that of the minimum inhibiting concentration for the particular organism to be treated.

Thus a subject of the invention is an active compound of the invention, such as mutactimycin PR, mutactimycin F, mutactimycin G or aldgamycin G, aldgamycin H or aldgamycin P10b for its use as a medicinal product.

A further subject of the invention is the use of a pharmaceutical composition according to the invention, for the production of an antibiotic for preventing and/or treating an infection implicating a gram positive bacterium, such as a streptococcus, a neonatal infection, a urinary infection, an endocarditis, a pneumonia, a meningitis, an otitis, a listeriosis, diphtheria, tuberculosis or leprosy.

A further subject of the invention is a method for treating and/or preventing an infection implicating a gram positive bacterium, such as a streptococcus, a neonatal infection, a urinary infection, an endocarditis, a pneumonia, a meningitis, and otitis, a listeriosis, diphtheria, tuberculosis or leprosy, by means of a pharmaceutical composition of the invention.

A further subject is the use of a pharmaceutical composition according to the invention, for the production of an antiviral medicinal product for preventing and/or treating an infection implicating the acquired immune deficiency syndrome (AIDS) virus, the vaccine virus, corona virus, papillomavirus, parvovirus, virus of catarrhal fever of sheep, dengue virus, Ebola virus, or influenza, smallpox, measles, rubella, varicella, hepatitis A, B, C, D or E, mononucleosis, yellow fever, encephalitis or herpes.

A further subject of the invention is a method for treating and/or preventing an infection of the acquired immune deficiency syndrome (AIDS) virus, the vaccine virus, the corona virus, papillomavirus, parvovirus, virus of catarrhal fever of sheep, dengue virus, Ebola virus, or influenza, smallpox, measles, rubella, varicella, hepatitis A, B, C, D, or E, mononucleosis, yellow fever, encephalitis or herpes, using a pharmaceutical composition of the invention.

A further subject of the invention is the use of a pharmaceutical composition according to the invention, for the production of an anticancer medicinal product for preventing and/or treating a subject suffering from a cancer, such as cancer of the lung, the uterus, breast or ovary, colorectal cancer, leukemia or a subject suffering from a tumour of the prostate, the bladder, skin, brain, throat.

A further subject of the invention is the method of treatment and/or prevention of a subject suffering from a cancer, such as cancer of the lung, the uterus, the breast, or the ovary, colorectal cancer, leukemia or a subject suffering from a tumour of the prostate, the bladder, the skin, brain, throat, using a pharmaceutical composition of the invention.

A further subject of the invention is a method for preventing as such and treating a disease in a plant using a phytopharmaceutical product comprising an active compound according to the invention, such as mutactimycin PR, mutactimycin F, mutactimycin G or aldgamycin G, aldgamycin H or aldgamycin P10b.

The active compounds of the invention such as mutactimycin PR, mutactimycin F, mutactimycin G or aldgamycin G, aldgamycin H or aldgamycin P10b can also be used for treatment in the food, leather or wood industry.

They are also useful in washing solutions for sanitary purposes, for example for washing hands, and for disinfecting various laboratory, dental and medical equipment or other contaminated materials.

The figure captions and examples below are intended to illustrate the invention without in any way limiting its scope.

### FIGURE CAPTIONS

FIG. 1: Fermentation profile of the strain Saccharothrix sp SA 103.

FIG. 2: Isolation and purification of the active products of the strain Saccharothrix sp SA 103.

### EXAMPLES

### Example 1

### Material and Methods

1.1. Microorganism

The productive actinomycete strain Saccharothrix SA 103 according to the invention was isolated from a soil of the arid regions of Algeria (Sahara) by placing a soil sample in suspension in sterile distilled water and deposition on a humic-vitamin B gelose containing 50 μg/ml of actidione. A pure culture of the strain SA 103 was preserved by freeze drying. It was also maintained at 6° C. for use in the laboratory on an ISP No. 2 inclined medium (Shirling and Gottlieb, Int. J. Syst. Bacteriol. 16:313-340, 1996).

1.2. Taxonomy

The taxonomic characteristics of the strain SA 103 were determined by culture on various media described in Shirling and Gottlieb (1996) and in Waksman (“The Actinomycetes”, Vol. II, The Williams, & Wilkins Co. Baltimore, 1961). The morphological characteristics were determined after growth at 30° C. for 14 days. The colour names and grade numbers were attributed using the ISCC-NBS system (“Inter Society Colour Council” for the “National Bureau of Standards”). The detailed observation of the morphologies of the mycelium and the spores was carried out by scanning electron microscopy (Hitachi, model S-450). The physiological properties were examined using the Goodfellow (J. Gen. Microbiol. 69:33-90, 1971) and Waksman (1961) methods. The type of isomers of diaminopimelic acid in the cell wall, and the total cellular composition of sugars were determined by the methods of Becker et al. (Appl. Microbiol. 12:421-423, 1964), and Lechevalier and Lechevalier (“In the Actinomycetales” Ed. H. Prauser, pp. 311-316, Fisher Verlag, Jena, 1970). Phospholipids and mycolic acids were analyzed by the procedure of Minnikin et al. (Int. J Syst. Bacteriol. 27:104-107, 1977; J. Chromatography 188:221-233, 1980).

1.3. Fermentation

A sample of the strain SA 103 taken from an immature inclined culture was inoculated into an Erlenmeyer flask containing 50 ml of the sterile culture medium containing 0.4t glucose, a 1% malt extract and a 0.4% yeast extract (adjusted to pH 7.2 before sterilisation) and placed in culture on a rotary stirrer (250 rpm) at 30° C. for two days. For producing antibiotics, 3 ml of culture medium were transferred to 500 ml Erlenmeyer flasks, each containing 100 ml of the above medium, and placed in culture for 10 days using the same conditions. The production of total antibacterial activity was carried out on a nutrient gelose, by a gelose diffusion test against Bacillus sutilis ATCC 6633. Inhibition of growth was examined after 24 hours of incubation at 30° C. Antimicrobial activity was estimated by measuring the diameter of the inhibition zone. The dry weight of the mycelium was determined in Eppendorf tubes filled with 1 ml of homogenized culture medium and dried at 105° C. for 24 hours (Pfefferle et al. J. Biotech. 80:135-142, 2000).

1.4. Purification of Antibiotics

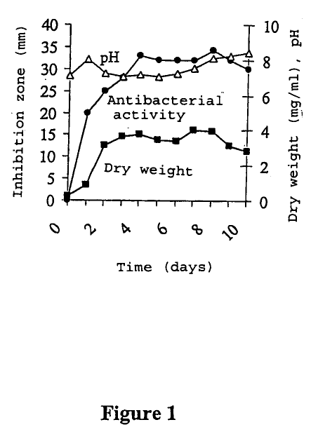

The production of antibacterial antibiotics by the strain SA 103 was carried out in liquid medium MS+starch (1%)+yeast extract (0.36) at pH 7.2 (100 ml of medium per 500 ml Erlenmeyer flask, stirring at 250 rpm, incubation at 30° C.). A kinetics showed that the strain SA 103 begins to produce antibiotics from the onset of the exponential phase (1^{st} day) and this production continues to the onset of the steady state phase before stabilising and then decreasing during the decline phase (cf. FIG. 1).

On the whole, eight litres of culture were prepared. The filtrates were extracted with n-butanol. The butanolic extract containing the desired antibacterial antibiotics was retained.

The butanolic extract, of a red colour, was concentrated on the Rotavapor. An aliquot of this extract was first sent through a Sephadex LH 20 column using methanol in double distilled water (80%) as the mobile phase. However, the separation of the antibiotics (visualisation by the naked eye and by the antibiography) was not conclusive. This step was therefore eliminated and the butanolic extract was purified directly by a reverse phase HPLC using a C18 column and isocratic conditions (63% methanol in water), a flow rate of 1.5 ml/min and detection at 220 nm. The fractions corresponding to all the peaks obtained on the profile were collected separately, concentrated and tested against Bacillus subtilis. Seven fractions (7 peaks) proved to be active: four of a bright red colour (P11, PR, F and G) and three colourless (P8, P10a and P10b). These antibiotics were purified by HPLC after 3 to 4 reinjections in the same conditions as previously.

### Example 2

### Results and Discussions—Taxonomic Characterisation of the Productive Strain

2.1. Morphology of the Strain

The strain SA 103 formed a well developed pink aerobic mycelium which broke into chains of straight or flexible spores. These spores were rod shaped and had a size of 1.9-2.9×0.6-0.7 microns with a smooth surface (FIG. 1). The endospores, scleral granules, synnemata and flagellate spores were not observed. The substrate mycelium was brownish red to deep red and showed few or no fragments. The strain produced a characteristic abundant dark red pigment which was revealed as corresponding to antibacterial antibiotics.

2.2. Structural Characteristics

Table 1 shows the culture characteristics of the strain SA 103 on various culture supports. The growth of the strain was abundant on yeast extract and malt extract gelose, Bennett gelose and nutrient gelose, but was moderate on oatmeal gelose and inorganic salts-starch gelose. The colour of the mycelium fit into the range between yellowish pink to light reddish brown for the aerobic hyphae and brownish orange to very dark red for the substrate mycelium. The strain produced a dark red or brownish orange soluble pigment on all the mediums used but no melanoid pigment was observed.

The least different species was Saccharothrix syringae which has a whitish-pinkish aerobic mycelium, a substrate mycelium and violet-red-brown soluble pigments.

2.3. Chemotaxonomy

The chemotaxonomic study showed the presence of meso-diaminopimelic acid and the absence of glycine (type III cell wall). The sugar motif of the overall cell consisted of rhamnose and galactose (type E cell sugar) (Kroppendstedt, “The genus Nocardiopsis. In the Procaryotes” Ed., A. Balows et al., pp. 1139-1159, Springer Verlag, Berlin, 1192)and the characteristic phospholipid was phosphatidyl-ethanolamine (phospholipids type PII). No mycolic acid was detected.

2.4. Physiology of the Strain

The results of the physiological tests are given Table 2.

The strain is capable of using several organic compounds (casein, gelatine, tween 80, starch, tyrosine, etc.) including most sugars. In contrast, with the exception of mannitol, the alcohol derivatives of the oses (inositol, adonitol, dulcitol, erythritol, and sorbitol) are not degraded. The strain is capable of growing at 20° C. and 48° C. (with an optimum at 30° C.) and at pH 5 and 9 (with an optimum between 7 and 8). It is resistant to the lysozyme and to crystal violet but is sensitive to all the antibiotics tested (11) with the exception of penicillin and rifampicin.

The strain SA 103 is distinguished from Sa. Syringae by its capacity to degrade lactose but not hypoxanthine and sodium butyrate, its sensitivity to erythromycin, gentamicin, oxytetracyclin, and vancomycin, its resistance to penicillin, rifampicin and crystal violet, and its growth at pH 5.

Accordingly, the strain SA 103 could be a novel species of Saccharothrix or possibly a novel subspecies of Sa. Syringae.

2.5. Classification

Based on the morphological and chemical properties described above, it was considered that the strain SA 103 belonged to the genus Saccharothrix (Labeda, et al, Int. J. Syst. Bacteriol. 34 :426-431, 1984). Compared to the nearest species Saccharothrix synrigae NRRL B-16 ₄₆₈^{T}, the strain SA 103 differed in its capacity to degrade lactose but not hypoxanthine and sodium butyrate, its susceptibility to erythromycin, gentamycin, oxytetracyclin and vancomycin, its resistance to penicillin, rifampicin, crystal violet and sodium azotide and the growth of pH 5.0. The strain was accordingly designated Saccharothrix sp. SA 103.

2.6. Fermentation

The variation over time of the production of antibacterial activity by Saccharothrix sp. SA 103 is shown in FIG. 1. The production of biological activity against B. subtilis began during the first day and reached its peak on day 4 and then became stable. The biomass increased during the first three days and remained stable, then decreased after day 8. The pH kinetics showed a striking increase during the first day, then became neutral, and rose at the end of fermentation. In general, the production of secondary metabolites by the microorganisms occurs during the steady state phase but in the present case, the production of biological activity was strongly correlated to growth and was observed throughout the variation over time. The same kinetics of production over time was observed for the production of dithiolopyrolone antibiotics by Saccharothrix sp. SA 233 (Lamari et al., J. Antibiotics 55:696-701, 2002) and the production of clavulanic acid by Streptomyces clavuligerus (Lebrihi et al., Appl. Microbiol. Biotechnol. 26:-130-135, 1987).

2.7. Determination of the Structure of the Antibiotics

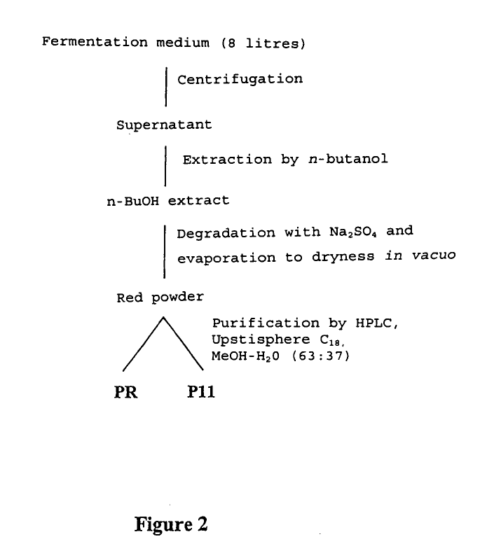

The various steps in isolation and purification of antibiotics are summarised in FIG. 2.

2.7.1. Antibiotics of the Anthracyclin Family

The antibiotics P11, PR, F and G are bright red in colour at neutral pH, yellow at acid pH and violet-blue at basic pH. P11 is the majority antibiotic and PR, the minority antibiotic. These antibiotics were the subject of the following rectoscopic analyses: UV visible light, infrared, mass spectrometry and NMR of the proton and carbon 13 (with studies of the correlations). Supplementary tests, such a solubility, determination of Rf by CCM and elemental analysis (the latter only for P11) were also carried out.

Identification of the P11 Molecule with Mutactimycin C

The P11 molecule has a molecular weight of 530 and the chemical formula C₂₇H₃₀O₁₁. The maxima obtained in the UV-visible (MeOH) are: 219, 234, 250, 287, 478, 496 and 531 mm. The spectrum is similar to that of the antibiotics of anthracycline family, and also the colour changes at different pH. The infrared spectrum suggests the presence of aromatics, hydroxyl, methyl and methoxyl groups. Moreover, the final structure was elucidated after a detailed study by NMR of the proton and of carbon 13 (chemical shifts, spin-spin coupling constants, signal intensities and ¹H—¹H cosy 45, ¹H—¹³C HMCQ and ¹H—¹³C HMBC correlations). The P11 molecule was thus identified as mutactimycin C, known to be secreted by a mutant of Streptomyces sp. Mutactimycin C consists of the anthracyclin nucleus (4 adjoining rings including a quinonic ring between two benzene rings), linked (via the carbon C7) to an ose (6-deoxy-3-O-methyl-a-mannopyranoside). P11 is highly soluble in water, methanol, n-butanol, ethanol, 1-propanol and acetone and insoluble in n-hexane and toluene.

Determination of the Chemical Structure of Novel Mutactimycins

Mutactimycin PR

The minor PR molecule has a molecular weight of 662 which corresponds to the formula C₃₂H₃₈O₁₅. The maxima obtained in the UV-visible and the infrared bands are identical to those of P11 and the mass fragments are fairly similar between the two antibiotics, suggesting a strong resemblance in the chemical structure. PR is highly soluble in water, methanol and ethanol and insoluble in chloroform, dichloromethane, ethyl acetate, n-hexane and toluene. The NMR of the proton and of carbon 13 served to elucidate the structure of PR which proved to be close to P11 (mutactimycin C) with the only difference being the presence of a second ose linked to the carbon for the 1^{st} benzene ring (in the place of —OCH₃). The second ose is 6-deoxy-mannopyranoside (difference from the first by the absence of a methyl) Due to the structure, the PR molecule belongs to the anthracyclin family and to the group of mutactimycins. However, it differs from all the known mutactimycins and hence represents a novel antibiotic of this group, called mutactimycin PR.

Mutactimycin F

The antibiotic F has a molecular weight of 516 (thus 14 less than P11) corresponding to a molecular formula C₂₆H₂₈O₁₁. Its UV-visible and infrared spectra and its mass fragments are very similar to those of P11 and PR. Hence, this suggests a strong resemblance between the three molecules. The F compound is soluble in water, n-butanol, 1-propanol and ethanol and insoluble in n-hexane and toluene. The NMR of the proton and the carbon 13 served to establish the structure of the molecule which proved to be very similar to P11 (mutactimycin C), with the sole difference of the presence of an OH on the carbon C3 of the mannopyranosyl residue, instead of a —OCH3 group. The F antibiotic is different from all known mutactimycins and represents a novel molecule called mutactimycin F.

Mutactimycin G

The molecule has a molecular weight of 502 corresponding to a formula C₂₅H₂₆O₁₁. Strong similarities between G, F, PR and P11 are observed in the UV-visible and infrared spectra, and the mass fragments. The G product is soluble in water, n-butanol, 1-propanol, methanol and ethanol and insoluble in chloroform, dichloromethane, ethyl acetate, n-hexane and toluene. The NMR of the proton and carbon 13 served to elucidate the structure of this molecule, which proved to be very similar to PR, with the only difference being the absence of a second ose linked to the carbon C7 of the 4^{th} ring of the anthracycline nucleus. The G antibiotic hence represents a novel molecule of the mutactimycin group and was therefore called mutactimycin G.

2.7.2. Antibiotics of the Macrolide Family

The antibiotics P8, P10a and P10b are not coloured. P8 is the majority molecule produced by Saccharothrix sp. SA 103 after P11 (mutactimycin C). The three antibiotics are highly soluble in methanol, n-butanol, ethanol, 1 and 2-propnol, ethyl acetate, acetone, chloroform and dichloromethane, but insoluble in water and n-hexane. These antibiotics were the subject of the same spectroscopic analyses as the mutactimycins described earlier, which served to culminate in the determination of their chemical structures.

Identification of P10a with Aldgamycin G

The P10a antibiotic has a molecular weight of 740 corresponding to the molecular formula C₃₇H₅₆O₁₅. The UV-visible spectrum is very similar to that of aldgamycins and showed a peak at 216 and two shoulders 245 and 278 nm. The infrared spectrum shows several absorption bands indicating the presence of methyl, methoxyl, hydroxyl groups and a carbonate function. P10a is soluble in n-butanol, 1 and 2-propanol, methanol, dichloromethane, ethyl acetate, chloroform, acetone and ethanol, and insoluble in n-hexane, toluene and water. The P10a molecule was thus identified with aldgamycin G, known to be secreted by Streptomyces avidinii. Aldgamycin G is a neutral macrolide whereof the lactonic ring comprises 16 atoms to which are connected two methylated sugars, mycinoe in position C14 and aldgarose in position C5.

Determination of the Chemical Structures of the Novel Macrolides

Aldgamycin H

The P8 antibiotic has a molecular weight of 714, thus a molecular formula C₃₆H₅₈O₁₄. Its physicochemical properties are very similar to those of P10a (aldgamycin G). However, the UV-visible spectrum of P10b shows a lower absorbance intensity at 245 and 278 nm and its infrared spectrum no longer contains the absorption band at 1800 cm⁻¹ characteristic of the carbonate group as in the case of aldgamycin G. The product P8, after analysis of all its spectroscopic data, was identified with a new aldgamycin called H which is similar to aldgamycin E, but differs in the absence of the carbonate function on the aldgarose sugar which is hydrolyzed in this case.

Swalpamycin B

The P10b molecule could not be separated from the P10a product by HPLC and accordingly, it was analysed at the same time as the product P10a in the form of a complex. It has a molecular weight of 698 and the chemical formula C₃₆H₅₈O₁₃. The NMR analyses of the proton and carbon 13 served to elucidate the final structure. Thus P10a has a chemical structure very similar to that of P8 and of swalpamycin.

Microbiological Activities of the Antibiotics

The microbiological activity of the mutactimycins P11, PR, F and G and the macrolides P8, P10 (a and b) is mainly directed against the gram positive bacteria. The most sensitive bacteria are Micrococcus luteus and Klebsiella pneumoniae which is the only gram negative bacteria to be sensitive.

The minimum inhibiting concentrations (MIC) are 5 μg/ml for M. luteus and K. pneumoniae, 10 pg/ml for Staphylococcus aureus CIP 53156, 40 μg/ml for Bacillus subtilis, 50 μg/ml for Listeria monocytogenes and 75 μl/ml for Mycobacterium smegmatis. The novel molecules PR, F and G are active against the same microorganisms (with the exception of L. monocytogenes).

None of the molecules have any action against S. aureus CIP 7625, the gram negative bacteria Escherichia coli, Pseudomonas syringae pathovar syringae and Agrobacterium tumefasciens and against the yeast Saccharomyces cerevisiae and the filamentous fungus Mucor romannianus.

The activity of the macrolides (aldgamycins G and H) and swalpamycin B is greater than those of the mutactimycins. In fact, the MIC are only 0.1 μg/ml for K. pneumoniae, 1 μg/ml for Bacillus subtilis, Micrococcus luteus and Staphylococcus aureus CIP 53156 which are the most sensitive and respectively 30 and 50 μg/ml for Listeria monocytogenes and Mycobacterium smegmatis. The gram negative bacteria (except K. pneumoniae) and the fungi are resistant.

| | MIC (μg/ml) |
|---|---|
| Test organisms | (swalpamycin) |
| Bacillus subtilis ATCC 6633 | 1 |
| Micrococcus luteus ATCC 9314 | 1 |
| Staphylococcus aureus CIP 7625 | >100 |
| Staphylococcus aureus CIP 53156 | 1 |
| Listeria monocytogenes CIP 82110 | 30 |
| Mycobacterium smegmatis ATCC 607 | 50 |
| Klebsiella pneumoniae CIP 82.91 | 0.1 |
| Escherichia coli ATCC 10536 | >100 |
| Pseudomonas syringae No 1882 | >100 |
| Agrobacterium tumefasciens No 2410 | >100 |
| Mucor ramannianus NRRL 1829 | >100 |
| Saccharomyces cerevisiae ATCC 4226 | >100 |

| | MIC (μg/ml) of |
|---|---|
| Test organisms | (aldgamycin H) |
| Bacillus subtilis ATCC 6633 | 10 |
| Micrococcus luteus ATCC 9314 | 1 |
| Staphylococcus aureus CIP 7625 | >100 |
| Staphylococcus aureus CIP 53156 | 5 |
| Listeria monocytogenes CIP 82110 | >100 |
| Mycobacterium smegmatis ATCC 607 | 20 |
| Klebsiella pneumoniae CIP 82.91 | 0.5 |
| Escherichia coli ATCC 10536 | >100 |
| Pseudomonas syringae No 1882 | >100 |
| Agrobacterium tumeffasciens No 2410 | >100 |
| Mucor ramannianus NRRL 1829 | >100 |
| Saccharomyces cerevisiae ATCC 4226 | >100 |

TABLE 1
Cultural Characteristics of Saccharothrix sp. SA 103.

| | | Aerobic | Substrate | Diffusible |
|---|---|---|---|---|
| Medium | Growth | Mycelium | Mycelium | Pigment |
| Yeast | Good | Abundant | Very dark | Blackish |
| extract-agar | | moderate | red (14) | red (16) |
| malt extract | | yellowish | | |
| (ISP No. 2) | | pink (29) | | |
| Agar oatmeal | Moderate | Moderate | Brownish | Brownish |
| flour (ISP | | pale | orange | orange |
| No. 3) | | yellowish | (54) | (54) |
| | | pink (31) | | |
| Inorganic | Moderate | Moderate | Brownish | Brownish |
| salts-agar | | brownish | orange | orange |
| starch (ISP | | pink (33) | (54) | (54) |
| No. 4) | | | | |
| Bennett agar | Good | Abundant | Dark | Dark |
| | | pale | reddish | reddish |
| | | reddish | brown (44) | brown (44) |
| | | brown (42) | | |
| Nutrient | Good | Moderate | Dark red | Dark red |
| gelose | | to | (13) | (13) |
| | | abundant | | |
| | | yellowish | | |
| | | pink (29) | | |

TABLE 2
Physiological Properties of the Strain
Saccharothrix sp. SA 103.

| | |
|---|---|
| Degradation of | − |
| Adenin | |
| Arbutin | + |
| Casein | + |
| Gelatin | + |
| Esculin | + |
| Guanine | − |
| Hypoxanthin | − |
| Starch | + |
| Testosterone | + |
| Tween 80 | + |
| Tyrosin | + |
| Xanthin | − |
| Adonitol | − |
| L-Arabinose | + |
| Cellobiose | + |
| Dextrin | + |
| Dulcitol | − |
| Erythritol | − |
| D-Fructose | + |
| Galactose | + |
| D-Glucose | + |
| Glycerol | + |
| Inositol | − |
| Lactose | − |
| Maltose | + |
| D-Mannitol | + |
| D-Mannose | + |
| Melezitose | − |
| Melibiose | − |
| α-Methyl-D-glucoside | − |
| D-Raffinose | − |
| L-Rhamnose | + |
| Ribose | + |
| Sorbitol | − |
| Saccharose | + |
| Trehalose | + |
| D-Xylose | + |
| Nitrate reduction | + |
| Production of melanoid | − |
| pigments | |
| Sodium decarboxylation: | + |
| Acetate | − |
| Benzoate | − |
| Butyrate | + |
| Citrate | − |
| Oxalate | + |
| Propionate | + |
| Pyruvate | + |
| Succinate | − |
| Tartrate | |
| Growth at: | + |
| 48° C. | + |
| pH 5 | + |
| pH 9 | |
| Tolerance to: | + |
| Crystal violet (0.001%) | + |
| Lysozyme (0.005%) | + |
| Phenol (0.05%) | − |
| Phenol (0.1%) | + |
| Potassium tellurite | + |
| (0.01%) | |
| Sodium azide (0.001%) | − |
| Sodium azide (0.01%) | − |
| Sodium chloride (5) | |
| Resistance to: | − |
| Chloramphenicol (25 μg/ml) | − |
| Cycloserin (10 μg/ml) | − |
| Erythromycin (10 μg/ml) | − |
| Gentamicin (10 μg/ml) | − |
| Kanamycin (25 μg/ml) | − |
| Novobiocin (10 μg/ml) | + |
| Oxytetracyclin (25 μg/ml) | + |
| Penicillin (25 μg/ml) | − |
| Rifampicin (5 μg/ml) | − |
| Streptomycin (10 μg/ml) | − |
| Vancomycin (5 μg/ml) | |

TABLE 3
Antimicrobial Spectrum of Antibiotics PR and P11:

MIC (μg/ml)

| | PR | P11 |
|---|---|---|
| Test strains | (1) | (2) |
| Bacillus subitlis ATCC 6633 | 75 | 40 |
| Micrococcus luteus ATCC 9314 | 50 | 5 |
| Staphylococus aureus CIP 7625 | >100 | >100 |
| Staphylococcus aureus CIP 53156 | 50 | 10 |
| Listeria monocytogenes CIP 82110 | >100 | 50 |
| Mycobacterium smegmatis ATCC 607 | >100 | 75 |
| Klebsiella pneumoniae CIP 82.91 | 40 | 5 |
| Escherichia coli ATCC 10536 | >100 | >100 |
| Pseudomonas syringae No 1882 | >100 | >100 |
| Agrobacterium tumefaciens No 2410 | >100 | >100 |
| Mucor ramannianus NRRL 1829 | >100 | >100 |
| Saccharomyces cerevisiae ATCC 4226 | >100 | >100 |

TABLE 4
Physicochemical Properties of the Products PR
and P11 (mutactimycin)

| | mutactimycin PR | P11 (mutactimycin C) |
|---|---|---|
| Appearance | Red powder | Red powder |
| Colour in H₂O | | |
| Acid | Yellow | Yellow |
| Neutral | Red | Red |
| Basic | Violet-blue | Violet-blue |
| Chemical formula | C₃₂H₃₈O₁₅ | C₂₇H₃₀O₁₁ |
| Molecular weight | 662 | 530 |
| Nano-ESI-MS (m/z) | | |
| Negative | 660.8 [M − H]⁻, | 528.8 [M − H]⁻, 351, |
| mode | 514.9, 354, 337, | 333, 315, 294.2, |
| | 319.1, 291.1 | 293.1 |
| Positive | 685.2 [M + Na]⁺ 539, | 553.1 [M + Na]⁺, 375, |
| mode | 507, 360.9, 342.9 | 356.9, 198.3 |
| Uvλₘₐₓnm in MeOH | 219, 234, 250, | 219, 234, 250, 287, |
| | 287, 478, 496, 531 | 478, 496, 531 |
| IRνₘₐₓ time in cell | 3393, 2969, 2932, | 3393, 2969, 2932, |
| (cm⁻¹) | 2878, 2841, 2709, | 2878, 2841, 2709, |
| | 2360, 2113, 1611, | 2360, 2113, 1611, |
| | 1583, 1444, 1408, | 1583, 1444, 1408, |
| | 1379, 1352, 1285, | 1379, 1352, 1285, |
| | 1238, 1213, 1133, | 1238, 1213, 1133, |
| | 1110, 1070, 1048 | 1110, 1070, 1048 |
| Relative | | |
| solubility | | |
| Highly | MeOH, EtOH, H₂O | MeOH, Me₂CO, H₂0, n- |
| soluble | | BuOH, EtOH, 1-PrOH |
| Medium | Me₂CO, n-BuOH, 1- | CH₂Cl₂, CHCl₃, EtOAc, |
| soluble | PrOH, 2-PrOH | 2-PrOH |
| Insoluble | CH₂Cl₂, CHCl₃, | n-hexane, toluene |
| | EtOAc, n-hexane, | |
| | toluene | |
| CCM (Value of Rf)^{a} | | |
| (I) | 0.16 | 0.44 |
| (II) | 0.58 | 0.64 |
| (III) | 0.82 | 0.80 |
| HPLC (Rt)^{b} | 31.74 min | 35.65 min |

^{a}CCM on silica gel (Merek No 5715. (I): EtOAc—MeOH (100:15). (II): n-BuOH—CH₃COOH—H₂O (3:1:1). (III): MeOH—CH₂Cl₂ (4:1).
^{b}HPLC conditions: Uptishpere C₁₈ UP5ODB (250 × 7.8 mm, i.d.), Mobile phase: isocratic at 63% MeOH in H₂O, Flow rate: 1.5 ml/min, Detection: UV (220 mm).

TABLE 5
Assignments of NMR data of ¹H and ¹³C of the
Product PR in DMSO-d6 at 298 K and in DMF-d₇ at 278 K.

| | |
|---|---|
| DMSO | DMF |

| Position | δH | δC | δH | δC |
|---|---|---|---|---|
| 1 | 8.00(d) | 121.0 | 7.96(d) | 120.6 |
| 2 | 7.84(br dd) | nd | 7.81(dd) | 135.3 |
| 3 | 7.66(br) | nd | 7.68(d) | 124.1 |
| 4 | | nd | | 157.9 |
| 4a | | nd | | 122.6 |
| 7 | 4.89(m) | 73.2 | 4.92(m) | 73.7 |
| 8 | 2.10(dd)/ | 43.3 | 2.16(dd)/ | 43.6 |
| | 1.92(dd) | | 1.98(dd) | |
| 9 | | 67.8 | | 67.7 |
| 10 | 2.83(d)/2.61(d) | 38.3 | 2.84(d)/ | 38.2 |
| | | | 2.65(d) | |
| 12a | | nd | | 136.0 |
| 6-OH | nd | | nd | |
| 9-OH | 4.70(br) | | nd | |
| 9-Me | 1.30(s) | 29.7 | 1.32(s) | 29.2 |
| 11-OH | nd | | nd | |
| 1′ | 5.16(br s) | 104.4 | 5.22(br | 104.7 |
| | | | s) | |
| 2′ | 3.83(m) | 67.0 | 3.92(m) | 67.1 |
| 3′ | 3.00(dd) | 81.5 | 3.04(dd) | 81.7 |
| 4′ | 3.33^{a} | 71.0 | 3.44(dd) | 72.0 |
| 5′ | 3.64(do) | 70.2 | 3.70^{a} | 69.8 |
| 2′-OH | 4.88 | | nd | |
| 3′-OMe | 3.22(s) | 56.9 | 3.17(s) | 56.6 |
| 4′-OH | 4.90 | | nd | |
| 5′-Me | 1.21(d) | 18.7 | 1.17(d) | 18.0 |
| 1″ | 5.69(br s) | 99.1 | 5.73(br | 99.4 |
| | | | s) | |
| 2″ | 4.00(m) | 71.0 | 4.09(m) | 71.3 |
| 3″ | 4.04(dd) | 70.9 | 4.10(dd) | 71.3 |
| 4″ | 3.35^{a} | 72.4 | 3.42(dd) | 72.2 |
| 5″ | 3.52(dq) | 70.9 | 3.54^{a} | 70.8 |
| 2″-OH | 5.15(br) | | nd | |
| 3″-OH | 4.89(br) | | nd | |
| 4″-OH | 4.97 | | nd | |
| 5″-Me | 1.09(d) | 18.7 | 1.05(d) | 17.9 |

The detector signals are represented.
^{a}Signal under residual HOD, nd: not detected.

TABLE 6
Assignments of NMR data of ¹H and ¹³C of product
P11 in DMSO-d6 at 298 K.

| Position | δH | δC |
|---|---|---|
| 1 | 7.81(m) | 119.4 |
| 2 | 7.83(m) | 136.0 |
| 3 | 7.56(m) | 119.1 |
| 4 | | 161.5 |
| 4a | | 120.8 |
| 5 | | 186.9 |
| 5a | | 111.1 |
| 6 | | 157.4 |
| 6a | | 136.0 |
| 7 | 4.86(dd) | 73.5 |
| 8 | 2.13(dd)/ | 42.8 |
| | 1.93(dd) | |
| 9 | | 68.2 |
| 10 | 2.76(d)/ | 37.4 |
| | 2.62(d) | |
| 10a | | 136.8 |
| 11 | | 155.4 |
| 11a | | 111.4 |
| 12 | | 187.4 |
| 12a | | 135.7 |
| 4-ONe | 3.95(s) | 56.2 |
| 6-OH | 14.13(s)^{a} | |
| 9-OH | 4.78(s) | |
| 9-Me | 1.31(s) | 28.1 |
| 11-OH | 13.19(s)^{a} | |
| 1′ | 5.12(d) | 104.2 |
| 2′ | 3.87(m) | 67.1 |
| 3′ | 3.02(dd) | 81.2 |
| 4′ | 3.32(ddd) | 71.7 |
| 5′ | 3.63(dq) | 69.8 |
| 2′-OH | 4.82(d) | |
| 3′-OMe | 3.23(s) | 56.1 |
| 4′-OH | 4.91(d) | |
| 5′-Me | 1.21(d) | 17.0 |

^{a}Signals which can be interchangeable

TABLE 7
Physicochemical Properties of the Compound F.

| | Mutactimycin F |
|---|---|
| Appearance | Bright red powder |
| Chemical formula | C₂₆H₂₈O₁₁ |
| Molecular weight | 516 |
| Nano-ESI-MS (m/z) | |
| Negative mode | 514.9 [M − H]⁻ |
| Positive mode | 539.11 [M + Na]⁺ |
| UVλₘₐₓ nm in MeOH | 219, 234, 252, 286, 473, 494, |
| | 531 |
| IRγₘₐₓ diamond cell (cm⁻¹) | 3351, 2970, 2927, 2854, 2360, |
| | 2336, 2114, 1981, 1798, 1610, |
| | 1582, 1444, 1406, 1381, 1356, |
| | 1271, 1240, 1215, 1137, 1091, |
| | 1069, 1048 |
| Relative solubility | |
| Highly soluble | n-BuOH, 1-PrOH |
| Medium soluble | MeOH, EtOH |
| Insoluble | H₂O, CH₂Cl₂, CHCl₃ |
| | EtOAc, Me₂CO, n-hexane, |
| | toluene |
| CCM (value of Rf)^{a} | |
| (I) | 0.30 |
| (II) | 0.62 |
| (III) | 0.78 |
| HPLC (Rt)^{b} | 22.25 min |

^{a}CCM on silica gel (Merch No 5715). (I): EtOAc—MeOH (100:15). (II): n-BuOH—CH₃COOH—H₂O (3:1:1). (III): MeOH—CH₂Cl₂ (4:1).
^{b}HPLC conditions: Uptisphere C₁₈ UP5ODB (250 × 7.8 mm, i.d.), Mobile phase: isocratic at 63% MeOH in H₂O, Flow rate: 1.5 ml/min, Detection: UV (220 nm).

TABLE 8
Assignments of NMR Data of ¹H and ¹³C of Product
F in DMSO-d6 at 298 K

| Position | δH | δC |
|---|---|---|
| 1 | 7.91(m) | 120.5 |
| 2 | 7.90(m) | 137.0 |
| 3 | 7.64(m) | 119.8 |
| 4 | | 161.6 |
| 4a | | 119.8 |
| 5 | | 187.4 |
| 5a | | 111.4 |
| 6 | | 157.5 |
| 6a | | 137.3 |
| 7 | 4.90(dd) | 73.2 |
| 8 | 2.13(dd)/ | 43.3 |
| | 1.94(dd) | |
| 9 | | 67.8 |
| 10 | 2.83(d)/ | 38.3 |
| | 2.64(d) | |
| 10a | | 137.0 |
| 11 | | 155.5 |
| 11a | | 111.5 |
| 12 | | 187.3 |
| 12a | | 135.7 |
| OMe-4 | 3.99(s) | 57.5 |
| OH-6 | 14.16(br | |
| | s)^{a} | |
| OH-9 | 4.78(s) | |
| Me-9 | 1.30(s) | 29.8 |
| OH-11 | 13.26(br | |
| | s)^{a} | |
| 1′ | 5.07(d) | 104.6 |
| 2′ | 3.61(m) | 71.4 |
| 3′ | 3.30(ddd) | 71.5 |
| 4′ | 3.24(ddd) | 72.7 |
| 5′ | 3.60(dq) | 70.2 |
| OH-2′ | 4.78(d) | |
| OH-3′ | 4.47(d) | |
| OH-4′ | 4.77(d) | |
| Me-5′ | 1.20(d) | 19.8 |

^{a}Signal under residual HOD.
nd: not detected

TABLE 9
Physicochemical Properties of Compound G.

| | G |
|---|---|
| Appearance | Bright red powder |
| Chemical formula | C₂₅H₂₆O₁₁ |
| Molecular weight | 502 |
| Nano-eSI-MS (m/s) | |
| Negative mode | 500.9 [M − H]⁻, 355, 337, |
| | 319.2, 291.2 |
| UVλₘₐₓ nm in MeOH | 219, 234, 252, 286, 473, 494, |
| | 531 |
| Irγₘₐₓ diamond cell | 3351, 2970, 2927, 2854, 2360, |
| | 2336, 2114, 1981, 1798, 1610, |
| | 1582, 1444, 1406, 1381, 1356, |
| | 1271, 1240, 1215, 1137, 1091, |
| | 1069, 1048 |
| Relative solubility | |
| Highly soluble | n-BuOH, l-PrOH |
| Medium soluble | MeOH, EtOH |
| Insoluble | H₂O, CH₂Cl₂, CHCl₃, EtOAc, |
| | Me₂CO, n-hexane, toluene |
| CCM (value of Rf)^{a} | |
| (I) | 0.30 |
| (II) | 0.62 |
| (III) | 0.78 |
| HPLC (Rt)^{b} | 22.24 min |

^{a}CCM on silica gel (Merch No 5715). (I): EtOAc—MeOH (100:15). (II): n-BuOH—CH₃COOH—H₂O (3:1:1). (III): MeOH—CH₂Cl₂ (4:1).
^{b}HPLC conditions: Uptisphere C₁₈ UP5ODB (250 × 7.8 mm, i.d.), Mobile phase: isocratic at 63% MeOH in H₂O, Flow rate: 1.5 ml/min, Detection: UV (220 nm).

TABLE 10
Assignments of NMR Data of ¹H and ¹³C of Product
G in DMSO-d6 at 298 K

| Position | δH | δC |
|---|---|---|
| 1 | 7.97(dd) | 120.9 |
| 2 | 7.87(dd) | 137.0 |
| 3 | 7.72(dd) | 124.2 |
| 4 | | 157.8 |
| 4a | | 121.2 |
| 5 | | nd |
| 5a | | 111.1 |
| 6 | | nd |
| 6a | | 139.6 |
| 7 | 5.08(dd) | 63.7 |
| 8 | 2.12(dd)/ | 44.3 |
| | 1.74(dd) | |
| 9 | | 68.1 |
| 10 | 2.73(br | 38.3 |
| | s) | |
| 10a | | 136.6 |
| 11 | | 155.4 |
| 11a | | 111.4 |
| 12 | | 186.2 |
| 12a | | 135.5 |
| OH-6 | 14.20(br | |
| | s)^{a} | |
| OH-7 | 4.89(s) | |
| OH-9 | 4.60(s) | |
| Me-9 | 1.31(s) | 30.7 |
| OH-11 | 13.30(br | |
| | s)^{a} | |
| 1′ | 5.65(br | 99.5 |
| | s) | |
| 2′ | 4.02(m) | 70.6 |
| 3′ | 4.02(m) | 71.0 |
| 4′ | 3.36(m) | 72.5 |
| 5′ | 3.55(dq) | 70.9 |
| OH-2′ | 5.15(d) | |
| OH-3′ | 4.86(d) | |
| OH-4′ | 4.96(d) | |
| Me-5′ | 1.10(d) | 19.8 |

^{a}Signal under residual HOD.
nd: not detected

TABLE 11
Assignments of NMR Data ¹H and ¹³c of the
Product P8(aldgamycin H) in CD₃OD and in DMSO-D₈ at 298 K

| | |
|---|---|
| P8(aldgamycin | P8(aldgamycin H) |
| H) in DMSO | in CD3OD |

| Position | dH(multi, J in Hz) | dH(multi, J in Hz) | dC |
|---|---|---|---|
| 1 | | | 166.4 |
| 2 | 6.09 d(15.3) | 6.03 d(15.4) | 120.9 |
| 3 | 6.56 dd(15.3; 10.6) | 6.64 dd(15.4; 10.6) | 152.1 |
| 4 | 2.72 ddq(10.6; | 2.83 ddq(10.6; | 41.9 |
| | 10.2; 6.8) | 10.2; 6.7) | |
| 5 | 3.27 br d(10.2) | 3.38 br d(10.2) | 86.8 |
| 6 | 0.98 br m | 1.14 br m | 34.6 |
| 7 | 1.40 ddd(14.4, | 1.59 ddd(14.0, | 32.0 |
| | 12.0, 4.5)/1.77 | 12.0, 4.1)/1.81 | |
| | ddd(14.4, 11.0, | ddd 14.0, 11.6, 3.1) | |
| | 3.4) | | |
| 8 | 2.41 ddq(12.0; 5.0; | 2.56 ddq(11.9; 4.4; | 45.2 |
| | 6.9) | 7.0 | |
| 9 | | | 202.8 |
| 10 | 7.02 d(15.4) | 6.92 d(15.4) | 126.7 |
| 11 | 6.20 dd(15.4; 9.4) | 6.35 dd(15.4; 9.4) | 144.1 |
| 12 | 3.43 dd(9.4; 2.0) | 3.43 dd(9.4; 1.9) | 59.0 |
| 13 | 2.99 dd(9.3; 2.0) | 3.06 dd(9.3; 1.9) | 59.2 |
| 14 | 1.37 br m | 1.42 br m | 49.8 |
| 15 | 5.21 dq(10.8; 6.3) | 5.40 dq(10.9; 6.2) | 68.7 |
| 16 | 1.25 d(6.3) | 1.36 d(6.2) | 17.4 |
| 17 | 1.15 d(6.8) | 1.26 d(6.7) | 18.4 |
| 18 | 0.89 d(7.5) | 1.02 d(6.8) | 16.6 |
| 19 | 1.10 d(6.9) | 1.18 d(7.0) | 16.8 |
| 20 | 3.59 dd(10.3; | 3.70 dd(10.1; | 67.1 |
| | 2.8)/3.94 dd(10.3, | 2.7/4.14 dd(10.1, | |
| | 2.9) | 2.9) | |
| 1′ | 4.39 d(8.0) | 4.60 d(7.9) | 103.0 |
| 2′ | 3.39 dd(8.0; 6.2) | 3.54 d(7.9) | 70.8 |
| 3′ | 1.32 dd(12.3; | 1.44 dd(11.8; | 75.4 |
| | 8.3)/1.38 dd(12.3; | 8.3)/1.55 dd(1.8; | |
| | 2.4) | 2.4) | |
| 4′ | | | 36.3 |
| 5′ | 3.73 ddq(8.3; 2.4; | 3.90 ddq(8.3; 2.4; | 66.6 |
| | 6.2) | 6.1) | |
| 6′ | 3.70 dq(6.3; 3.9) | 3.90 q(6.5) | 68.5 |
| OH-2′ | 4.75 d(6.2) | nd | |
| OH-3′ | 3.89 br s | nd | |
| Me-5′ | 1.07 d(6.2) | 1.18 d(6.1) | 20.4 |
| OH-6′ | 4.47 d(3.9) | nd | |
| Me-6′ | 0.98 d(6.3) | 1.17 d(6.5) | 15.7 |
| 1″ | 4.49 d(8.1) | 4.60 d(8.0) | 101.2 |
| 2″ | 3.02 dd(8.1; 2.7) | 3.11 dd(8.0; 2.9) | 81.8 |
| 3″ | 3.66 dd(2.7; 2.6) | 3.80 dd(2.9; 2.8) | 80.5 |
| 4″ | 3.09 ddd(9.6; 7.1; | 3.20 ddd(9.5; 2.8) | 73.6 |
| | 2.6) | | |
| 5″ | 3.54 dq(9.6; 6.2) | 3.69 dq(9.5; 6.3) | 70.1 |
| OMe-2″ | 3.41 s | 3.57 s | 58.5 |
| OMe-3″ | 3.46 s | 3.60 s | 61.1 |
| OH-4″ | 4.88 d(7.1) | nd | |
| Me-5″ | 1.12 d(6.2) | 1.25 d(6.3) | 17.1 |

nd: not detected

TABLE 12
Physicochemical Properties of Products P8 and P10a

| | P8 | P10a |
|---|---|---|
| Appearance | Colorless powder | Colorless powder |
| Chemical formula | C₃₆H₅₈0₁₄ | C₃₇H₅₆0₁₅ |
| Molecular weight | 714 | 740 |
| Nano-ESI-MS (m/z) | | |
| Positive | 737.4 [M + Na]⁺, | 763.44 [M + Na]⁺, |
| mode | 563.3, 545.2, | 589.27., 563, |
| | 389.2, 371.1, 289.1 | 571.24, 559.19, |
| | | 545.29, 417.16, |
| | | 389.16, 371.16, |
| | | 289.02 |
| UVλₘₐₓ nm | 216.73, shoulder | 216.73, shoulder |
| in MeOH | 245.71 and 280 | 245.71 and 278.09 |
| IRγₘₐₓ diamond cell | 3431, 2970, 2931, | 3361, 2968, 2920, |
| (cm⁻¹) | 2880, 1712, 1689, | 2851, 1799, 1712, |
| | 1654, 1620, 1581, | 1653, 1623, 1593, |
| | 1454, 1416, 1382, | 1562, 1508, 1457, |
| | 1354, 1326, 1279, | 1417, 1383, 1356, |
| | 1263, 1236, 1172, | 1324, 1282, 1238, |
| | 1159, 1117, 1082 | 1194, 1173, 1159, |
| | | 1117, 1084, 1047 |
| Relative solubility | | |
| Highly | MeOH, EtOR, Me₂CO, | MeOH, EtOH, Me₂CO, |
| Soluble | n-BuOH, 1-PrOH, 2- | n-BuOH, 1-PrOH, 2- |
| | PrOH, CH₂Cl₂, | PrOH, CH₂Cl₂, |
| | EtOAc, CHCl₃ | EtOAc, CHCl₃ |
| Insoluble | n-hexane, toluene, | n-hexane, toluene, |
| | H₂O | H₂O |
| CCM (value of Rf)^{a} | | |
| (I) | 0.63 | 0.68 |
| (II) | 0.71 | 0.74 |
| (III) | 0.86 | 0.88 |
| HPLC (Rt)^{b} | 23.21 min | 28.38 min |

^{a}CCM on silica gel (Merch No 5715). (I): EtOAc—MeOH (100:15). (II): n-BuOH—CH₃COOH—H₂O (3:1:1). (III): MeOH—CH₂Cl₂ (4:1).
^{b}HPLC conditions: Uptisphere C₁₈ UP5ODB (250 × 7.8 mm, i.d.), Mobile phase: isocratic at 63% MeOH in H₂O, Flow rate: 1.5 ml/min, Detection: UV (220 nm).

TABLE 13
Assignments of NMR Data ¹H and ¹³C of the
Product P10a (aldgamycin G) in CD₃OD at 298K

P10a (aldgamycin G)

| Position | δH (Multi, J in Hz) | δC |
|---|---|---|
| 1 | | 166.3 |
| 2 | 6.03 d (15.5) | 121.0 |
| 3 | 6.64 dd (15.5; 10.7) | 151.8 |
| 4 | 2.84 ddq (10.7; 10.2; 6.8) | 41.8 |
| 5 | 3.42 d (10.2) | 86.8 |
| 6 | 1.16 m | 34.5 |
| 7 | 1.56 m/1.82 ddd (14.0; 11.8, 2.9) | 31.9 |
| 8 | 2.54 ddq (11.8; 4.8; 7.0) | 45.2 |
| 9 | | 202.7 |
| 10 | 6.92 d (15.4) | 126.6 |
| 11 | 6.35 dd (15.4; 9.4) | 144.2 |
| 12 | 3.43 dd (9.4; 2.1) | 59.0 |
| 13 | 3.06 dd (9.3; 2.1) | 59.2 |
| 14 | 1.43 dm (10.8) | 49.8 |
| 15 | 5.40 dq (10.8; 6.3) | 68.7 |
| 16 | 1.36 d (6.3) | 17.4 |
| 17 | 1.24 d (6.8) | 18.2 |
| 18 | 1.01 d (6.8) | 16.6 |
| 19 | 1.18 d (7.0) | 16.8 |
| 20 | 3.70 dd (10.2, 3.1)/4.13 dd (10.2; 2.29) | 67.1 |
| 1′ | 4.59 d (7.7) | 102.4 |
| 2′ | 3.47 d (7.7) | 70.6 |
| 3′ | | 86.6 |
| 4′ | 1.61 dd (14.6; 11:1)/1.92 dd (14.6; 2.1) | 41.1 |
| 5′ | 3.84 ddq (11.1; 6.1; 2.1) | 67.0 |
| 6′ | 4.50 q (6.5) | 82.2 |
| 7′ | | 156.6 |
| OH-2′ | nd | |
| Me-5′ | 1.23 d (6.1) | 19.9 |
| Me-6′ | 1.57 d (6.5) | 12.6 |
| 1″ | 4.60 d (8.0) | 101.2 |
| 2″ | 3.11 dd (8.0; 2.8) | 81.8 |
| 3″ | 3.80 dd (2.8; 2.7) | 80.5 |
| 4″ | 3.20 dd (9.6; 2.7) | 73.6 |
| 5″ | 3.69 dq (9.6; 6.2) | 70.1 |
| OMe-2″ | 3.57 s | 58.5 |
| OMe-3″ | 3.60 s | 61.1 |
| OH-4″ | nd | |
| Me-5″ | 1.25 d (6.2) | 17.1 |

nd: not detected

TABLE 14
Assignments of NMR Data ¹H and ¹³C of the
Product P10b (swalpamycine B) in CD₃OD at 298K

P10b (swalpamycine B)

| Position | δH (Multi, J in Hz) | δC |
|---|---|---|
| 1 | | 166.7 |
| 2 | 5.88 d (15.4) | 121.4 |
| 3 | 6.62 dd (15.4; 10.0) | 152.5 |
| 4 | 2.79 m | 41.3 |
| 5 | 3.36 br d (10.1) | 87.3 |
| 6 | 1.21 m | 34.5 |
| 7 | 1.59 m^{a}/1.70 ddd (14.4, 11.7, 3.2) | 31.9 |
| 8 | 2.53 m | 45.3 |
| 9 | | 205.7 |
| 10 | 6.46 d (15.0) | 123.9 |
| 11 | 7.08 dd (15.0; 10.9) | 142.4 |
| 12 | 6.28 dd (15.2; 10.9) | 133.4 |
| 13 | 6.08 dd (15.2; 9.3) | 142.0 |
| 14 | 2.46 m | 51.5 |
| 15 | 5.12 dq (10.1; 6.3) | 69.7 |
| 16 | 1.39 d (6.8) | 17.6 |
| 17 | 1.22 d (6.3) | 19.1 |
| 18 | 1.01 d (6.8) | 16.9 |
| 19 | 1.18 d (7.0) | 17.0 |
| 20 | 3.66 dd (9.9; 3.6)/4.01 dd (9.9, 3.4) | 68.5 |
| 1′ | 4.56 d (8.0) | 102.9 |
| 2′ | 3.54 d (8.0) | 70.8 |
| 3′ | | 75.4 |
| 4′ | 1.46 m/1.54 m | 36.3 |
| 5′ | 3.87 ddq (8.3; 6.2; 2.4) | 66.6 |
| 6′ | 3.90 q (6.6) | 68.5 |
| OH-2′ | nd | |
| OH-3′ | nd | |
| Me-5′ | 1.19 d (6.2) | 20.4 |
| OH-6′ | nd | |
| Me-6′ | 1.16 d (6.6) | 15.6 |
| 1″ | 4.61 d (8.0) | 101.3 |
| 2″ | 3.08 dd (8.0; 2.9) | 81.8 |
| 3″ | 3.77 dd (2.9; 2.8) | 80.6 |
| 4″ | 3.19 dd (9.6; 2.9) | 73.6 |
| 5″ | 3.67 dq (9.6; 6.3) | 70.0 |
| OMe-2″ | 3.53 s | 58.6 |
| OMe-3″ | 3.59 s | 61.1 |
| OH-4″ | nd | |
| Me-5″ | 1.24 d (6.3) | 17.1 |

nd: not detected

1-25. (canceled)

26. Actinomycete strain Saccharothrix SA registered at CNCM on 16 Feb. 2004 under number I-3160 or a mutant strain thereof.

27. Method for selecting the actinomycete strain Saccharothrix SA 103 according to claim 26 and/or at least one of its mutant strains, characterized in that it comprises the following steps:
a) contacting of a biological sample likely to contain the said strain and/or at least one of its mutant strains with an appropriate selection medium;
b) isolation of the said strain and/or at least one of its mutant strains.

28. Method for producing a culture medium from a culture of the actinomycete strain Saccharothrix SA 103 and/or at least one of its mutant strains according to claim 26, characterized in that it comprises the following steps:
a) fermentation of the said strain in a nutrient medium to obtain the culture medium;
b) optionally, separation of the culture medium obtained in step a).

29. Method for producing a culture medium according to claim 28, characterized in that the separation carried out in optional step b) is a centrifugation and/or a filtration and/or a pasteurization.

30. Culture medium capable of being obtained by the method according to claim 28.

31. Method for producing an active concentrate from the culture medium according to claim 30, characterized in that it comprises the following steps:
a) organic extraction of the culture medium with an organic solvent;
b) optionally, dehydration of the organic phase obtained and/or drying in vacuo;
c) optionally, placing of the active concentrate in suspension, preferably filtration of the suspension obtained, arid repetition of the steps a) and b) of organic extraction and dehydration.

32. Active concentrate capable of being obtained by the method of claim 31.

33. Method for producing an active compound from the active concentrate according to claim 32 by reverse phase high performance liquid chromatography (reverse phase HPLC), preferably preceded by thin layer chromatography and/or low pressure liquid chromatography.

34. Method for producing an active compound according to claim 33, characterized in that the active compound is a mutactimycin such as mutactimycin P11, mutactimycin PR, mutactimycin G or mutactimycin F, or an aldgamycin such as aldgamycin G, aldgamycin H or aldgamycin P10b, or the pharmaceutically acceptable addition salts, isomers, enantionmers, diastereoisomers, and mixtures of these active compounds.

35. Active compound capable of being obtained by the production method according to claim 33, characterized in that the active compound is mutactimycin PR having the following formula: 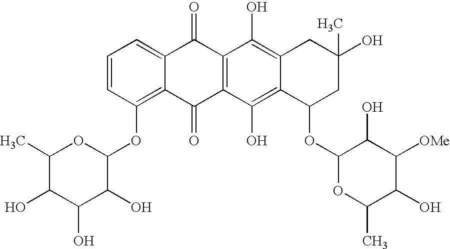
or its pharmaceutically acceptable addition salts, isomers, enantiomers, diastereoisomers, and mixtures thereof.

36. Active compound capable of being obtained by the production method according to claim 33, characterized in that the active compound is mutactimycin F having the following formula: 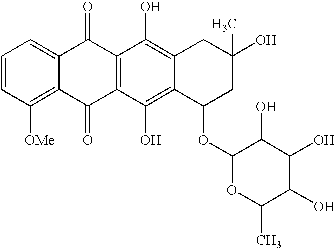
or its pharmaceutically acceptable addition salts, isomers, enantiomers, diastereoisomers, and mixtures thereof.

37. Active compound capable of being obtained by the production method according to claim 33, characterized in that the active compound is mutactimycin G having the following formula: 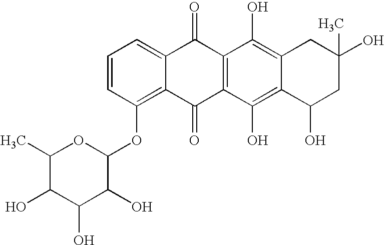
or its pharmaceutically acceptable addition salts, isomers, enantiomers, diastereoisomers, and mixtures thereof.

38. Active compound capable of being obtained by the production method according to claim 33, characterized in that the active compound is aldgamycin G having the following stereochemical formula: 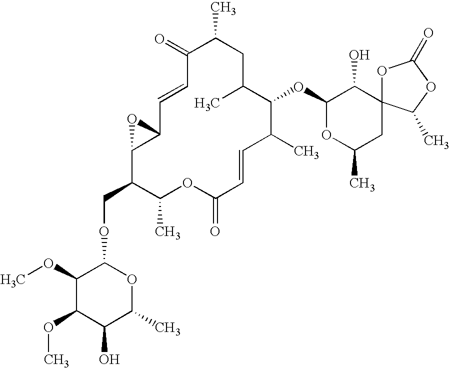
or its pharmaceutical acceptable addition salts, isomers, enantiomers, diastereoisomers, and mixtures thereof.

39. Active compound capable of being obtained by the production method according to claim 33, characterized in that the active compound is aldgamycin H having the following formula: 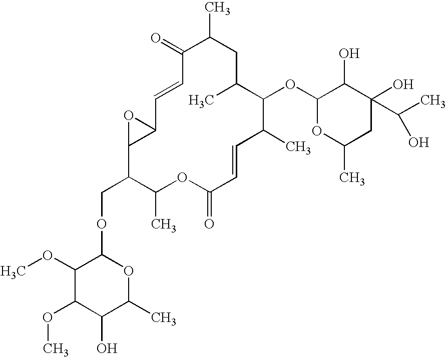
or its pharmaceutically acceptable addition salts, isomers, enantiomers, diastereoisomers, and mixtures thereof.

40. Active compound according to claim 39, characterized in that the aldgamycin H has the following stereochemical formula: 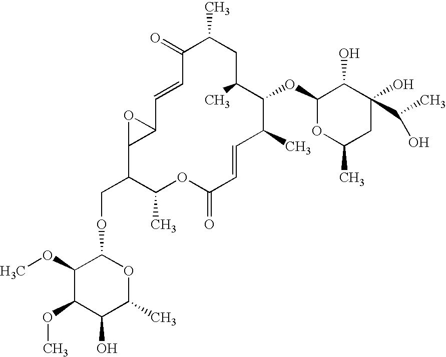
or its pharmaceutical acceptable addition salts, isomers, enantiomers, diastereoisomers, and mixtures thereof.

41. Active compound capable of being obtained by the production method according to claim 33, characterized in that the active compound is aldgamycin P10b having the following formula: 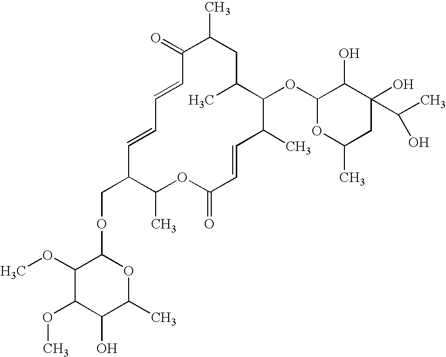
or its pharmaceutically acceptable addition salts, isomers, enantiomers, diastereoisomers, and mixtures thereof.

42. Active compound according to claim 41, characterized in that the aldgamycin P10b has the following stereo chemical formula: 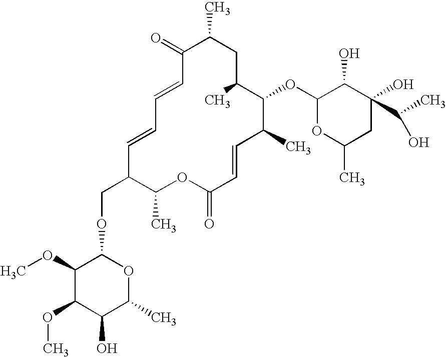
or its pharmaceutical acceptable addition salts, isomers, enantiomers, diastereoisomers, and mixtures thereof.

43. Pharmaceutical composition containing a therapeutically effective quantity of an active compound according to claim 35 and a pharmaceutical acceptable excipient.

44. Active compound according to claim 35 for its use as a medicinal product.

45. Method for the production of an antibiotic for preventing and/or treating an infection implicating a gram positive bacterium, such as a streptococcus, a neonatal infection, a urinary infection, an endocarditis, a pneumonia, a meningitis, an otitis, a listeriosis, diphtheria, tuberculosis or leprosy, comprising an effective amount of a pharmaceutical composition according to claim 43.

46. Method for the production of an antiviral medicinal product preventing and/or treating an infection implicating the acquired immune deficiency syndrome (AIDS) virus, the vaccine virus, corona virus, papillomavirus, parvovirus, virus of catarrhal fever of sheep, dengue virus, Ebola virus, or influenza, smallpox, measles, rubella, varicella, hepatitis A, B, C, D or E, mononucleosis, yellow fever, encephalitis or herpes, comprising an effective amount of a pharmaceutical composition according to claim 43.

47. Method for the production of an anticancer medicinal product for preventing and/or treating a subject suffering from a cancer, such as cancer of the lung, the uterus, breast or ovary, colorectal cancer, leukemia or a subject suffering from a tumour of the prostate, the bladder, skin, brain, throat, comprising an effective amount of a pharmaceutical composition according to claim 43.

48. Method for preventing or treating a disease in a plant using a phytopharmaceutical product comprising an active compound according to claim 35.
